(19) United States
(12) Patent Application Publication
(10) Pub. No.: US 2025/0134727 A1
(43) Pub. Date: May 1, 2025

(54) Panty Replacement Worn With Outerwear

(71) Applicant: Skimpies, Inc., Dover, DE (US)

(72) Inventor: Margaret Bentley Santa Maria, Los Angeles, CA (US)

(21) Appl. No.: 18/385,072

(22) Filed: Oct. 30, 2023

(51) Int. Cl. A61F 13/472; A61F 13/47

(52) U.S. Cl. CPC A61F 13/472; A61F 2013/4708

(57) ABSTRACT

In one embodiment, a panty replacement includes one or more cotton layers, each layer comprising a first substantially rounded end, a second substantially rounded end opposite the first substantially rounded end along a length of the product, and a tapered middle portion coupled to the first substantially rounded end and the second substantially rounded end, wherein a width of the tapered middle portion is less than a width of the first substantially rounded end and is less than a width of the second substantially rounded end. The panty replacement further includes an adhesive layer that forms a bottom surface of the panty replacement. The adhesive layer is coupled to a bottom surface of the one or more cotton layers and configured to removably affix the panty replacement to a crotch area of an outerwear garment.

### TECHNICAL FIELD

This application generally relates to a panty replacement worn with outerwear.

### BACKGROUND

Feminine hygiene products include sanitary pads and panty liners, among others. Pads and panty liners are both typically used to absorb blood from the vagina, but typically serve somewhat different purposes in achieving those ends. For example, pads tend to be thicker, heavier products used in circumstances in which blood discharge is relatively greater, and panty liners tend to be thinner, lighter products than pads, and are used in circumstance in which blood discharge is relatively lighter. In use, both pads and panty liners are designed to sit within a panty, which is covered by an outerwear garment such as pants, shorts, leggings, etc.

Panties are undergarments of a variety of styles that are worn between a woman's skin and an outer garment, such as pants, shorts, or leggings. Unlike pads or panty liners, panties are not typically intended to absorb blood, but rather serve hygienic and/or comfort purposes. For example, a panty may keep sweat or other liquid off of an outer garment, and may serve as a barrier between stiffer materials (e.g., denim) and the skin.

### BRIEF DESCRIPTION OF THE DRAWINGS

FIG. 1 illustrates an example panty replacement that provides a number of benefits described herein.

FIG. 2 illustrates a specific embodiment of the example panty replacement of FIG. 1.

### DESCRIPTION OF EXAMPLE EMBODIMENTS

Feminine hygiene products include sanitary pads and panty liners, among others. Pads and panty liners are both typically used to absorb blood from the vagina, but typically serve somewhat different purposes in achieving those ends. For example, pads tend to be thicker, heavier products used in circumstances in which blood discharge is relatively greater, and panty liners tend to be thinner, lighter products than pads, and are used in circumstance in which blood discharge is relatively lighter.

Both pads and panty liners are placed inside of panties, and are not panty replacements. Panties are undergarments that are worn underneath another garment, such as shorts, pants, or leggings. Like panty liners, pads typically are designed to sit within panties. Some pads and panty liners have “wings,” or strips of material extending from roughly the middle of each side of the pad or panty liner, which are intended to secure the pad or panty liner to the panty by wrapping around the gusset, or crotch, portion of the panty garment. Other pads or panty liners are secured to a panty garment by a sticky material that covers an interior portion of the pad or panty liner or sticks the pad or panty liner to the gusset of the panty garment.

Panties are undergarments of a variety of styles that are worn between a woman's skin and an outer garment, such as pants, shorts, or leggings. Unlike pads or panty liners, panties are not typically intended to absorb blood, but rather serve hygienic and/or comfort purposes. For example, a panty may keep sweat or other liquid off of an outer garment, and may serve as a barrier between stiffer materials (e.g., denim) and the skin.

However, panties present a number of downsides in many use cases. For example, under tight-fitting outerwear, such as leggings or other outerwear made of elastic material, panties create visible protrusions (e.g., lines, bulges, etc.) through the outerwear. These protrusions include an outline of the panty, and are often aesthetically undesirable. These protrusions occur even for relatively low-profile panties, such as thongs, for example because the band of the panty or other portions of the panty create bulk that results in visible protrusions through the leggings. In addition, such protrusions vary in visibility as a result of movement; e.g., a panty that may have somewhat low-visibility protrusion when sitting can result in high visibility protrusions when moving, such while walking, running, or doing exercises. These movement-based protrusions are often exacerbated because exercise outergarments tend to be relatively thin, tight, and flexible, such as leggings. As mentioned above, even panties that are designed to be relatively low profile (e.g., thongs, panties that stick to the skin, etc.) suffer from these problems.

However, outwear is not designed to by a panty replacement, and forgoing underwear under outerwear also results in undesirable issue. For example, panties serve as a barrier that helps keep outerwear clean, and not wearing panties results in either suboptimal hygiene or having to wash outerwear more frequently than is desirable. Washing often degrades outerwear and its performance characteristics (e.g., the elasticity of leggings), and washing also reduces the useful life of outerwear. However, not washing outerwear when worn without panties can result reduced cleanliness (e.g., discharge collecting on the inside of the outerwear) and may result in increased likelihood of undesirable smells and/or medical conditions (e.g., urinary tract infections). In addition, wearing outerwear without panties increases the likelihood that the outerwear should be washing in relatively hot water, which further reduces the performance characteristics and useful life of the outerwear, and may alter the outerwear's overall characteristics (e.g., its size or shape etc.). Finally, outwear may be made of fabric that is uncomfortable when worn directly against the skin (e.g., rougher fabrics, such as denim) and may include chemicals that can cause skin reactions or are not designed to be placed directly against the skin, especially for an extended period of time.

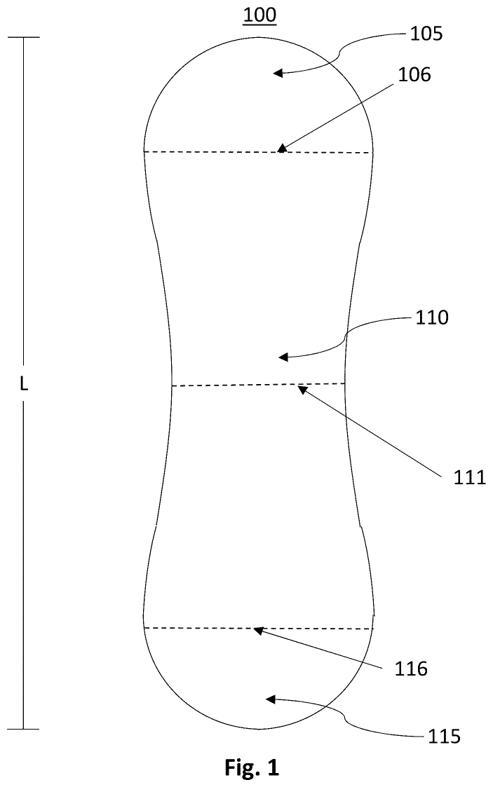

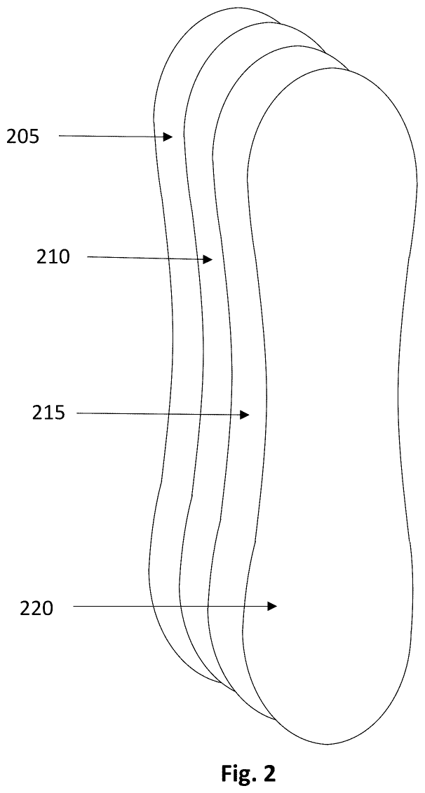

FIG. 1 illustrates an example panty replacement that provides a number of benefits described herein. FIG. 2 illustrates a specific embodiment of the example panty replacement of FIG. 1. As described more fully herein, the panty replacement may be specifically sized, shaped, and designed to adhere to a crotch area of outerwear, providing comfort and protection without resulting in visible protrusions.

The example of FIG. 1 provides an overhead view looking down at the top surface of panty replacement 100. Panty replacement 100 includes a first substantially rounded end 105. At the other end of panty replacement 100 (i.e., at the other end opposite the length L of panty replacement 100) is a second substantially rounded end 115. Substantially rounded end 105 and substantially rounded end 115 are joined by a tapered middle section 110. The length and shape of panty replacement 100 results in a panty replacement that covers the crotch area of a woman's body, absorbing discharge and sweat, while protecting sensitive regions (e.g., the vagina and vulva) from rubbing against the outerwear. While example panty replacement 100 illustrates rounded end 105 and rounded end 115 as being symmetric (both in size and in shape), this disclosure contemplates that the two ends need not be symmetric in either size or shape. As one example, end 105 and end 115 may have slightly different sizes, even though both ends may have the same shape.

In the example of FIG. 1, end 105 and end 115 are illustrated as being rounded throughout the length of their edges. However, this disclosure contemplates that a panty replacement may have substantially rounded ends that includes rounding along only a portion of the edges of those ends. For example, an end may be substantially rounded by a rounded section that connects a straight portion of the edge of the panty replacement with another straight portion of the edge of the panty replacement. For example, with reference to FIG. 1, the top and bottom edges of the panty replacement may be straight, and the ends may be rounded at the corners (i.e., rounding that connects a top straight edge to the side edges of the panty replacement and that connects a bottom straight edge to the side edges of the panty replacement). While the discussion above of the example of FIG. 1 describes the ends as being substantially rounded, this disclosure contemplates that a panty replacement may have a different shape, e.g., may have rectangular ends (or ends of another shape) without rounding. Moreover, while the discussion above of the example of FIG. 1 describes the middle section as being tapered (and FIG. 1 illustrates a particular, example shape of that taper), this disclosure contemplates that a panty replacement may not be tapered in the middle section. For example, a panty replacement may have a rectangular or substantially rectangular shape throughout its body.

As shown in example panty replacement 100, substantially rounded end 105 has a maximum width 106, and substantially rounded end 115 has a maximum width 116. In particular embodiments, width 106 and 116 may be the same, although this disclosure contemplates that the maximum widths may be different. Tapered middle portion 110 has a minimum width 111. In particular embodiments, panty replacement 100 may be shaped so that a ratio of minimum width 111 to width 106 and/or to width 116 has a fixed value. For example, a ratio of width 111 to width 106 may be 75%, which defines the degree of tapering from the first end 105 to the middle section, and from the middle section to the second end 115. This specific “wide-tapered-wide” shape helps increase surface area of the panty replacement (increasing adhesion, as described below), helps ensure coverage of sensitive areas that may produce some discharge (e.g., urine or vaginal discharge), and avoids bunching in the relatively narrower crotch region of the body. In addition, this shape also ensures the panty replacement lays flat, without bunching, in outerwear, such as leggings, and this shape allows the panty replacement to move with the body and outerwear while providing panty coverage over the entire crotch area. In particular embodiments width 106 and width 116 may be approximately 2 inches, while width 111 may be approximately 1.5 inches, although this disclosure contemplates other absolute and relative widths.

As shown in the example of FIG. 1, panty replacement 100 has a length L. In particular embodiments, L may be approximately 6 inches, although this disclosure contemplates that other sizes may be used. In particular embodiments, a ratio of length L to width 106 and/or to width 116 may be a fixed value, e.g., a value of approximately 3. In other words, as the size L is changed, in particular embodiments width 106 and/or 116 (and, in particular embodiments, width 111) may change proportionally.

FIG. 2 illustrates an example embodiment of the panty replacement of FIG. 1 that consists of four layers. Layer 220, or the top layer of the panty replacement, contacts the woman's skin while in use. In particular embodiments, layer 220 is made of a cotton material, such as an organic cotton material. Organic cotton may decrease the likelihood of inducing skin reactions (e.g., rashes), and cotton is a breathable, soft, and somewhat absorbent material, increasing hygiene, comfort, and protection of the outerwear. In particular embodiments, layer 220 may be a woven or nonwoven cotton (e.g., spunlace).

In particular embodiments, a panty replacement may be a 2-ply construction, e.g., two layers of organic cotton. In particular embodiments, a panty replacement may be a 1-ply construction. In addition, thicker panty replacements (e.g., a panty replacement having more plies of cotton material) may be used; however, decreased thickness improves comfort, reduces bunching, and ensures that the panty replacement will not result in visible protrusions through the outerwear. Thus, in particular embodiments, one or two plies may be preferred.

In the example of FIG. 2, layer 210 is a backing layer, or bottom layer, that provides a suitable surface on which to apply adhesive layer 205, which is described in more detail below. In particular embodiments, layer 210 may be a PLA layer, and may be corn-based and/or made of other biodegradable materials. In particular embodiments, layer 210 may include perforations or micro needling through the layer to increase the breathability of the panty replacement by allowing air to pass through layer 210. In particular embodiments, perforations or micro needling may occur in regions of layer 210 that are interspersed between adhesive regions of layer 205 (e.g., adhesive layer 205 may be spaced strips applied on layer 210, and perforations or micro needling may be applied in regions of layer 210 between the spaced strips). In particular embodiments, perforations or micro needling in layer 210 may also pass through other layers. For example, perforations or micro needling may pass through layers 215 and 220 and/or through layer 205.

As illustrated in the example of FIG. 2, layers 210 and 220 are connected by a thin adhesive layer 215. This disclosure contemplates that adhesive layer 215 may be of any suitable type, such as a hot-melt adhesive. Layer 215 may be an air-bonded layer or an H3-bonded layer, in particular embodiments. In particular embodiments, layer 215 may not include any absorbent material (e.g., stuffed cotton fibers), which are commonly found in adhesive layers of panty liners or pads to absorb vaginal discharge, but that are not necessary for the panty replacements described herein, which serve different purposes than pads and panty liners.

Layer 205 is a thin adhesive layer that forms the bottom of the panty replacement. Adhesive layer 205 may be a pressure-sensitive adhesive that removably adheres the panty replacement to the crotch area of an outerwear. In particular embodiments, layer 205 may be covered by a backing that can be removed (e.g., peeled off) by the user when putting the panty replacement into use. For example, a user may peel a cover off of layer 205 and then press layer 205 into the crotch area of an outerwear, creating a strong but removeable bond between the outerwear and the panty replacement. The strength of the adhesion between the panty replacement and the outerwear is stronger than the adhesive force between the skin and top layer 220; i.e., the panty replacement stays affixed to, and moves with, the outerwear even when there is some adhesive force (e.g., due to friction, moisture, etc.) between layer 220 and the skin. Thus, the panty replacement provides the sensation of having only outerwear on, except with the added comfort and protection of the panty replacement.

In particular embodiments, adhesive layer 205 may be smaller than layers 210-220, which may have the same size. In particular embodiments layer 205 may cover at least all of the bottom of the tapered middle portion of layer 210, as this entire coverage increases the adhesion of the panty replacement to the outerwear and reduces folding and bunching of the panty replacement, ensuring that no visible lines appear through the outerwear. In particular embodiments, adhesive layer 205 may be the same size as layer 210, i.e., layer 205 may cover the entire bottom (all the way to the edge) of layer 210. In particular embodiments, adhesive layer 205 may cover all but a small portion of the edge of one of the ends of layer 210 (e.g., all but a small portion of end 105 or of end 115), for example in order to assist the user in peeling a cover off of adhesive layer 205 when the user is ready to use the panty replacement. When affixed to outerwear, adhesive layer 205 can be removed by pulling the panty replacement off of the outerwear. The panty replacement comes off in a single piece while leaving no adhesive residue (or leaving substantially no adhesive residue) on the outerwear.

In particular embodiments, adhesive layer 205 may have a different shape than the layers above (e.g., a different shape than layer 210). For example, layer 205 may be a number of horizontal strips of adhesive material, with spaces between the strips. In particular embodiments, each strip may cover the width of the panty replacement (i.e., may be present from edge to edge of the panty replacement). In particular embodiments, other shapes or patterns other than strips may be used. In particular embodiments, the width of each strip of adhesive may be equal to the space between strips, or may have a different dimension (e.g., there may be more or less space between strips than the width of one strip).

In particular embodiments, layers 205-220 may be dye-free, e.g., in order to increase the compatibility of the panty replacement with human skin. In particular embodiment, layer 220 or other layers may include dyes for coloring purposes.

In particular embodiments, a thickness of the panty replacement (e.g., as shown in FIG. 1 and FIG. 2) is constant throughout the surface area of the panty replacement. For example, the thickness of the panty replacement at the very edge of the panty replacement may be the same as the thickness of the panty replacement in the center portions of the panty replacement. In particular embodiments, layer 220 may adhere tightly to layer 210 (and/or layer 210 may adhere tightly to layer 205) such that the top layer of the panty replacement has no “waves” or “ripples” or other bunching of material across its top surface, creating a smooth and even top surface. In particular embodiments, the top layer of the panty replacement (e.g., layer 220 in the example of FIG. 2) may have no stitching or other lines or depressions, resulting in a smooth, cotton surface that does not bunch for contacting the skin.

As explained above, the panty replacements described herein may be used with any suitable outwear, including but not limited to leggings, body suits, skinny jeans, swimsuits, pantyhose, shorts (such as linen shorts), etc. The panty replacement adheres directly to the outwear and moves with the outerwear, largely creating the sensation of wearing only the outerwear, but with the added comfort of the panty replacement, which is often a softer and more comfortable material than the outerwear is made of. In addition, as described above, the panty replacement protects the outerwear from discharge and sweat while absorbing at least some of those fluids from the user's skin. In addition, the panty replacement removes the need for panties entirely and avoids bunching or folding, maintaining a smooth surface that conforms to the user's skin while avoiding protrusions through the outerwear-even outerwear that is relatively thin and tight (e.g., leggings). Moreover, as described above, particular embodiments of the panty replacements described herein improve comfort and promote healthy skin by being breathable, for example by having a breathable bottom layer with perforations or micro needling that pass through the layer and/or through the panty replacement product.

As described herein, embodiments of this disclosure are an item of apparel known as a panty replacement or leggings liner that works as a panty solely over the crotch area and affixes directly to outerwear such as leggings or jeans. Embodiments of this item of apparel include a disposable panty replacement worn in cases in which women may previously have gone commando in the past, due to the tightness of their outwear garment, such as leggings or tight pants and shorts. Embodiments described herein provide the comfort, breathability, and protection of a cotton panty over the crotch area with no sides or waistband to the panty replacement, and therefore, such embodiments leave no lines or bulges on the body.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A panty replacement comprising:
one or more cotton layers, each layer comprising a first substantially rounded end, a second substantially rounded end opposite the first substantially rounded end along a length of the product, and a tapered middle portion coupled to the first substantially rounded end and the second substantially rounded end, wherein a width of the tapered middle portion is less than a width of the first substantially rounded end and is less than a width of the second substantially rounded end; and
an adhesive layer forming a bottom surface of the panty replacement, the adhesive layer coupled either directly or indirectly to the one or more cotton layers and configured to removably affix the panty replacement to a crotch area of an outerwear garment.

2. The panty replacement of claim 1, wherein the one or more cotton layers comprise one or two cotton layers.

3. The panty replacement of claim 1, further comprising a backing layer coupled between the one or more cotton layers, wherein the adhesive layer couples directly to a bottom surface of the backing layer.

4. The panty replacement of claim 3, wherein the backing layer comprises one or more perforated regions or micro-needled regions.

5. The panty replacement of claim 4, wherein the bottom surface of the backing layer comprises one or more regions that are covered by the adhesive layer and one or more regions that are not covered by the adhesive layer, and wherein the perforated regions or micro needled regions are located in regions of the backing layer that are not covered by the adhesive layer.

6. The panty replacement of claim 5, wherein the perforated regions or micro needled regions pass through the one or more cotton layers.

7. The panty replacement of claim 1, wherein the one or more cotton layers comprise an organic cotton material.

8. The panty replacement of claim 1, wherein the adhesive layer comprises a pressure-sensitive adhesive.

9. The panty replacement of claim 1, wherein the outerwear garment comprises leggings.

10. The panty replacement of claim 1, wherein a ratio of the length to a largest width of the first substantially rounded end is about 3.

11. The panty replacement of claim 10, wherein the length of the product is approximately six inches.

12. The panty replacement of claim 1, wherein a largest width of the first substantially rounded end is the same as a largest width of the second substantially rounded end.

13. The panty replacement of claim 12, wherein a length from a smallest width of the middle tapered portion to the largest width of the first substantially rounded end is the same as a length from the smallest width of the middle tapered portion to the largest width of the second substantially rounded end.

14. The panty replacement of claim 13, wherein a ratio of the smallest width of the substantially tapered middle portion to the largest width of the first substantially rounded end is about 0.75.

15. The panty replacement of claim 14, wherein the smallest width of the substantially tapered middle portion is about 1.5 inches.

16. The panty replacement of claim 1, wherein the adhesive layer covers the entire bottom surface of the tapered middle portion of the one or more cotton layers.

17. The panty replacement of claim 16, wherein the adhesive layer covers the entire bottom surface of the one or more cotton layers.

18. The panty replacement of claim 16, wherein the adhesive layer covers substantially all of the entire bottom surface of the one or more cotton layers, except for a small region at the edge of the first substantially rounded end or a small region at the edge of the second substantially rounded end.

19. The panty replacement of claim 1, wherein the adhesive layer comprises a plurality of adhesive strips, wherein the adhesive strips are separated by a space.

20. The panty replacement of claim 19, wherein plurality of adhesive strips run from a first edge of the panty replacement to a second edge of the panty replacement.
